(19) United States
(12) Patent Application Publication
(10) Pub. No.: US 2017/0095630 A1
(43) Pub. Date: Apr. 6, 2017

(54) Oxygen Port Nasal Cannula

(71) Applicant: Wake Forest University Health Sciences, Winston-Salem, NC (US)

(72) Inventor: Robert Patrick Yeatts, Winston-Salem, NC (US)

(21) Appl. No.: 15/314,779

(22) PCT Filed: May 28, 2015
(86) PCT No.: PCT/US15/33025

§ 371 Date: Nov. 29, 2016

Related U.S. Application Data
(60) Provisional application No. 62/004,950, filed on May 30, 2014.

(51) Int. Cl. A61M 16/06; A61B 5/097; A61B 5/083; A61M 16/04; A61M 16/08

(52) U.S. Cl. CPC A61M 16/0672; A61M 16/042; A61M 16/0461; A61M 16/085; A61B 5/0836; A61B 5/097; A61M 2016/0413; A61M 2230/432

(57) ABSTRACT

A device for use in delivering oxygen to a human patient while measuring carbon dioxide exhaled from that patient, comprises: (a) a base member having an oxygen delivery inlet port and a carbon dioxide detection outlet port formed therein separate from the oxygen delivery port; (b) a flexible cannula extending from the base, the cannula having a proximal end portion, an intermediate portion, a distal end portion, a first lumen formed therein, and a second lumen separate from the first lumen formed therein, with the distal end portion connected to the base member, the first lumen in fluid communication with the oxygen delivery inlet port, and the second lumen in fluid communication with the carbon dioxide detection outlet port.

### RELATED APPLICATIONS

This application claims the benefit of U.S. provisional patent application Ser. No. 62/004,950, filed May 30, 2014, the disclosure of which is incorporated by reference herein in its entirety.

### FIELD OF THE INVENTION

The present invention concerns nasal oxygen supply cannula for human subjects, and methods of using the same.

### BACKGROUND OF THE INVENTION

In the operative suite, patients undergoing an operation under heavy local sedation are administered supplemental oxygen to support cardiopulmonary function. Respiratory rate and tidal volumes are frequently reduced as the result of anesthetic agents and analgesic medication administered during the course of operation. Oxygen supplementation is commonly delivered by nasal cannula or by use of an anesthetic mask. End-tidal carbon dioxide, a measure of pulmonary function, is also monitored providing information regarding respiratory rate and serves as a measure of lung function.

A double lumen nasal cannula is the most common method for delivery of supplemental oxygen with simultaneous detection of expiratory carbon dioxide. These cannulas are fit around the ear and check, and rest on the upper lip. The cannulas have short ports or prongs that insert into the nose. While these devices function well in many circumstances, they are problematic for surgery that involves the the head and neck by interfering with access to facial structures. The design is also problematic in patients who are mouth breathers, or patients undergoing deep local sedation when obstruction of the nasopharynx can occur from analgesic or anesthetic agent delivery. Shallow breathing or low tidal volumes, an effect of heavy sedation, reduce alveolar oxygen tension as a result of inadequate gas exchange. This may be compounded by inadequate oxygen delivery despite attempts at supplemental oxygen delivery.

Dislocation of the nasal prongs and their associated oxygen ports can also be problematic with nasal cannulas currently in use. Supplemental oxygen delivery may not be delivered as designed, as the nasal prongs may be easily dislocated out of the nose. Such dislocation can be hazardous as oxygen blown into a surgical field where cauterizing instruments are used is a well known cause of operating room explosion, fires and patient thermal burns. See, e.g., S. Mehta et al., Operating Room Fires: A Closed Claims Analysis, Anesthesiology 118, 1133-9 (2013); S. Hart et al., Operating Room Fire Safety, The Oschner J 11, 37-42 (2011).

### SUMMARY OF THE INVENTION

A device for use in delivering oxygen to a human patient while measuring carbon dioxide exhaled from that patient is provided herein. The device comprises (a) a base member having an oxygen delivery inlet port and a separate carbon dioxide detection outlet port formed therein; and (b) a flexible cannula extending from the base. The cannula has a proximal end portion, an intermediate portion, a distal end portion, with the distal end portion connected to the base member. The cannula has a first lumen formed therein, and a second lumen separate from the first lumen formed therein. The proximal end portion has at least one oxygen delivery outlet port formed therein (e.g., for positioning in the nasopharynx), and the intermediate portion has at least one carbon dioxide detection inlet port formed therein (e.g., for positioning in the nostril or “nasal vestibule” of the patient). The first lumen is in fluid communication with both the oxygen delivery inlet port and the oxygen delivery outlet port, and the second lumen in fluid communication with both the carbon dioxide detection outlet port and the carbon dioxide detection inlet port

The foregoing and other objects and aspects of the present invention are explained in greater detail in the drawings herein and the specification set forth below.

### BRIEF DESCRIPTION OF THE DRAWINGS

FIG. 1 is a schematic diagram of an embodiment of the present invention.

FIG. 2 is a second diagram of the embodiment of FIG. 1, rotated ninety degrees.

FIG. 3 is a sectional view of a portion of a human head and neck showing portions of the embodiment of FIGS. 1-2 in place for use in a patient or subject.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Like numbers refer to like elements throughout. In the figures, the thickness of certain lines, layers, components, elements or features may be exaggerated for clarity. Where used, broken lines illustrate optional features or operations unless specified otherwise.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, may be used herein for ease of description to describe an element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus the exemplary term “under” can encompass both an orientation of over and under. The device may otherwise be oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only, unless specifically indicated otherwise.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the present invention. The sequence of operations (or steps) is not limited to the order presented in the claims or figures unless specifically indicated otherwise.

Patients or subjects in which the present invention may be used are, in general, human subjects, including both male and female subjects, and including neonate, infant, juvenile, adolescent, adult, and geriatric subjects.

“Proximal” as used herein to describe portions of the present invention is with respect to the patient (e.g., closer to or internal to the patient) when the device or apparatus is in use, rather than with respect to the components of the device or apparatus that are external to the patient when the device or apparatus is in use (which may be more distant from, or “distal to” the patient).

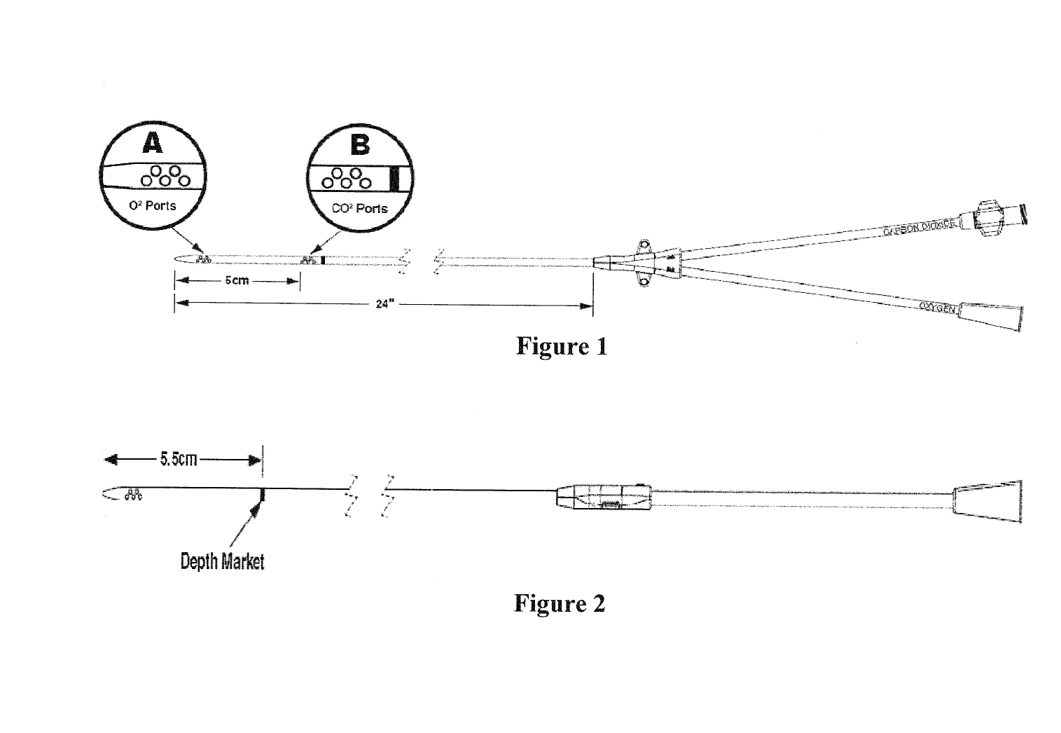

A non-limiting embodiment of the present invention is illustrated in FIGS. 1 to 3. As illustrated in FIGS. 1-2, the device comprises (a) a base member having an oxygen delivery inlet port and a separate carbon dioxide detection outlet port formed therein; delivery port; and (b) a flexible cannula extending from the base. The cannula has a proximal end portion, an intermediate portion, a distal end portion, with the distal end portion connected to the base member. The cannula has a first lumen formed therein, and a second lumen separate from the first lumen formed therein.

The proximal end portion has at least one oxygen delivery outlet port formed therein (e.g., for positioning in the nasopharynx, particularly the posterior nasopharynx, of the patient), and the intermediate portion has at least one carbon dioxide detection inlet port formed therein (e.g., for positioning in a nostril or nasal vestibule of the patient). The first lumen is in fluid communication with both the oxygen delivery inlet port and the oxygen delivery outlet port, and the second lumen in fluid communication with both the carbon dioxide detection outlet port and the carbon dioxide detection inlet port.

The proximal end portion has, in some embodiments, a blunt configuration or blunt tip, (e.g., fully or partially rounded, elliptical, flat with beveled edge portions, etc.) to facilitate non-traumatic insertion through a nostril and nasal vestibule and into the nasopharynx of a patient. The oxygen delivery port(s) are preferably positioned in the side wall of the proximal end portion, though in other embodiments the oxygen delivery port(s) may be positioned on the blunt tip of the proximal end portion.

There is at least one indicia (e.g., a line, letter, or the like) formed and/or marked on the cannula, positioned a predetermined distance from said cannula proximal end portion, and configured to aid a user in visually positioning said proximal end portion in the nasopharynx of a patient. Two or more such indicia may optionally be included to indicate a range of depth insertions, e.g., a maximum or minimum range, and/or different ranges for different size patients). The specific dimensions of the device and location of indicia and ports may vary depending upon the size, weight, age, and/or gender of the subject for which the apparatus is intended. In general, the at least one indicia on the cannula distal end portion is spaced from 4 or 5 to 6 or 7 centimeters from the proximal terminus of the cannula (e.g., the blunt tip), and/or from about 0.1, 0.2, or 0.4, to 0.6, 0.8 or 1 centimeters from the at least one carbon dioxide detection inlet port. In general in an adult subject, the at least one oxygen delivery outlet port is spaced about one-half centimeter from the proximal terminus of the cannula (e.g., said blunt tip), and/or from 4 or 5 to 6 or 7 centimeters from the at least one carbon dioxide detection inlet port, and/or from 4 or 5 to 6 or 7 centimeters from the at least one indicia (when the indicia is included on the cannula distal end portion.

Specific dimensions are given for one illustrative embodiment in FIGS. 1-2, but other dimensions may be used for different size patients as discussed above and below.

The first lumen has, in some embodiments, a cross-section area or average diameter not less than (e.g., greater than or equal to) that of said second lumen. The second lumen need not extend the entire length of the cannula beyond the carbon dioxide detection inlet ports, though such extension may optionally be provided when manufacture is simplified).

In general, while the oxygen delivery inlet port consists of a single inlet port, the at least one oxygen delivery outlet port may comprise a single or a plurality (e.g., 3, 5, 6 or 8 or more) outlet ports in fluid communication with the first lumen. A plurality of ports are preferred (whether positioned on the side wall or blunt tip of the end portion) to allow more diffuse delivery of oxygen. Similarly, while the carbon dioxide detection outlet port consists of a single outlet port; the at least one carbon dioxide inlet port may comprise a single or a plurality (e.g., 3, 5, 6 or 8 or more) inlet ports in fluid communication with the second lumen. The oxygen delivery outlet ports and the carbon dioxide inlet ports may be positioned on the same side as one another (depending on the internal arrangement of the lumens, e.g., parallel, partially spiral, etc.) or may be positioned on opposite sides with respect to one another, depending on the internal arrangement of the lumens). In some embodiments, the oxygen delivery outlet ports are distributed circumferentially around the cannula proximal end portion, for example, in case one side abuts the wall of the nasopharynx or the soft palate and the ports on that side partially blocked or occluded.

The cannula may be made of any suitable flexible polymer material, such as surgical-grade silicone polymer, and may have any suitable diameter, for example, 6, 7, 8, 9 or 10 French. In general, the cannula has a length of 20, 25, or 35 centimeters, up to 45 or 50 centimeters or more, again depending on the size of the subject.

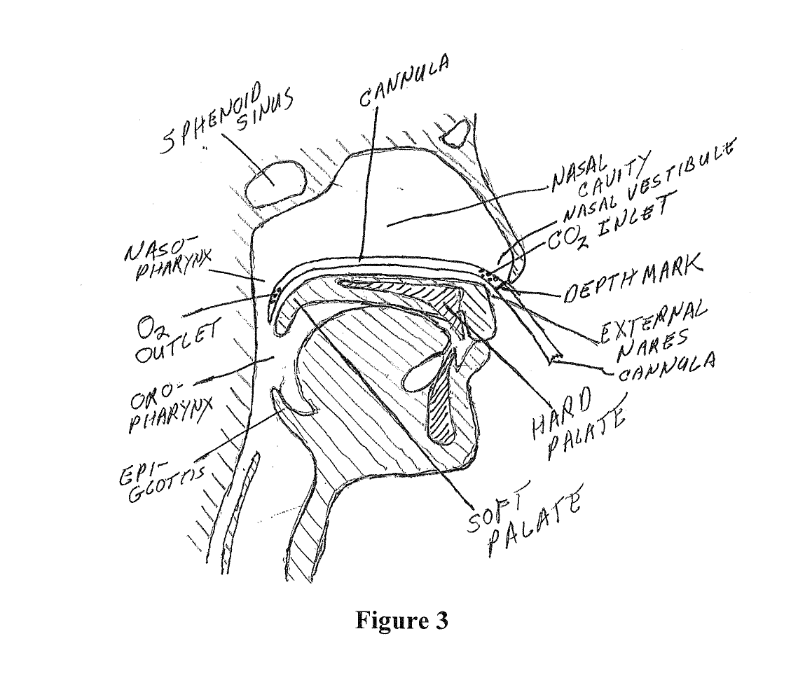

As shown in FIG. 3, the cannula is dimensioned so that the proximal tip portion is insertable through a nostril and nasal vestibule and into the nasopharynx, preferably the posterior nasopharynx, of a patient, with the at least one carbon dioxide detection inlet port positioned in the nostril or nasal vestibule of the patient and the base member positioned outside the patient when the at least one oxygen delivery outlet port is positioned in the nasopharynx, preferably posterior nasopharynx, of that patient.

In use, the cannula is inserted through a nostril of the patient, positioned (e.g., with the aid of the indicia noted above) as described above, a patient carbon dioxide monitor (not shown) operably connected to the carbon dioxide detection outlet port, and an oxygen supply (not shown) operably connected to the oxygen delivery inlet port. The cannula distal portion, and/or the body, may be secured to the patient's upper lip, cheek or other region with surgical tape, adhesive, or other fastener to minimize movement thereof. Any suitable oxygen supply or carbon dioxide monitor may be used, as described below.

Oxygen supply. Any suitable mobile or immobile patient oxygen supply or source can be used, including humidified and non-humidified oxygen sources such as hospital-based oxygen supplies, bottled oxygen or pressurized canisters of oxygen, oxygen gn concentrators such as the Inogen 1 G2 Oxygen Concentrator (available from Medical Industries America), etc., with optional back-up oxygen sources, pressure regulators, humidifiers and the like. See, e.g., U.S. Pat. No. 8,307,825.

Carbon dioxide monitors. Any suitable patient carbon dioxide monitor (typically an end-tidal carbon monoxide monitor) Numerous examples are available, including but not limited to the Capnocheck PLUS capnograph, Capnocheck SLEEP capnograph, and Capnocheck II Hand-Held capnograph (available from BCI/Smith Medical); the RespSense capnograph and the LifeSense capnograph (available from Nonin Medical), the EMMA capnometer (available from Phasein); the CO₂ SMO capnograph (available from COSMO); the ECHO CO₂ capnograph (available from DR); etc. See also U.S. Pat. No. 4,423,739.

The foregoing is illustrative of the present invention, and is not to be construed as limiting thereof. The invention is defined by the following claims, with equivalents of the claims to be included therein.

1. A device for use in delivering oxygen to a human patient while measuring carbon dioxide exhaled from that patient, comprising:
(a) a base member having an oxygen delivery inlet port and a carbon dioxide detection outlet port formed therein separate from said oxygen delivery port;
(b) a flexible cannula extending from said base, said cannula having a proximal end portion, an intermediate portion, a distal end portion, a first lumen formed therein, and a second lumen separate from said first lumen formed therein, with said distal end portion connected to said base member, said first lumen in fluid communication with said oxygen delivery inlet port, and said second lumen in fluid communication with said carbon dioxide detection outlet port; with
said proximal end portion having a blunt configuration to facilitate non-traumatic insertion through a nostril and nasal vestibule into the nasopharynx of a patient, and having at least one oxygen delivery outlet port in fluid communication with said first lumen;
said intermediate portion having at least one carbon dioxide detection inlet port formed therein in fluid communication with said second lumen; and
said cannula so dimensioned that said proximal tip portion is insertable through a nostril and nasal vestibule and into the nasopharynx of a patient, with said at least one carbon dioxide detection inlet port positioned in the nasal vestibule of the patient and said base member positioned outside the patient when said at least one oxygen delivery outlet port is positioned in the nasopharynx of that patient.

2. The device of claim 1, said proximal end portion having a side wall and a blunt tip, with said oxygen delivery outlet port formed in said side wall.

3. The device of claim 2, wherein said blunt tip comprises a rounded tip.

4. The device of claim 1, further comprising:
(c) at least one indicia on either said base member or on said cannula distal end portion, positioned a predetermined distance from said cannula proximal end portion, and configured to aid a user in visually positioning said proximal end portion in the nasopharynx of a patient.

5. The device of claim 4, wherein said at least one indicia is on said cannula distal end portion and is spaced from 4 or 5 to 6 or 7 centimeters from a proximal terminus of said cannula; and/or about from about 0.1, 0.2 or 0.4 to 0.6, 0.8 or 1 centimeter from the at least one carbon dioxide detection inlet port.

6. The device of claim 1, wherein said at least one oxygen delivery outlet port is placed at a proximal terminus of the cannula and/or alternatively is spaced about one-half centimeter from the proximal terminus of said cannula, and/or from 4 or 5 to 6 or 7 centimeters from said at least one carbon dioxide detection inlet port, and/or from 4 or 5 to 6 or 7 centimeters from said at least one indicia when said indicia is on said cannula distal end portion.

7. The device of claim 1, wherein said first lumen has a cross-section area or average diameter not less than that of said second lumen.

8. The device of claim 1, wherein:
said oxygen delivery inlet port consists of a single inlet port, and
said carbon dioxide detection outlet port consists of a single outlet port;

9. The device of claim 1, wherein: said at least one oxygen delivery outlet port comprises a plurality of outlet ports in fluid communication with said first lumen.

10. The device of claim 1, wherein said at least one carbon dioxide detection inlet port comprises a plurality of inlet ports in fluid communication with said second lumen.

11. The device of claim 1, wherein said cannula is comprised of a flexible polymer material.

12. The device of claim 1, wherein said cannula has a diameter of 6, 7, 8, 9 or 10 French.

13. The device of claim 1, wherein said cannula has a length of 35 to 45 centimeters.

14. An apparatus for delivering oxygen to a human patient while measuring carbon dioxide exhaled from that patient, comprising:
(a) a device of claim 1; and
(b) a patient carbon dioxide monitor operably connected to said carbon dioxide detection outlet port.

15. The apparatus of claim 14, further comprising:
(c) an oxygen supply operably connected to said oxygen delivery inlet port.
